(19) United States
(12) Patent Application Publication
(10) Pub. No.: US 2024/0074735 A1
(43) Pub. Date: Mar. 7, 2024

(54) POINT OF CARE ULTRASOUND AS A TOOL TO ASSESS WOUND SIZE AND TISSUE REGENRATION AFTER SKIN GRAFTING

(71) Applicant: THE REGENTS OF THE UNIVERSITY OF CALIFORNIA, Oakland, CA (US)

(72) Inventors: Jesse JOKERST, La Jolla, CA (US); Yash Mantri, La Jolla, CA (US)

(21) Appl. No.: 18/261,524

(22) PCT Filed: Jan. 14, 2022
(86) PCT No.: PCT/US2022/012433

§ 371 Date: Jul. 14, 2023

Related U.S. Application Data
(60) Provisional application No. 63/137,275, filed on Jan. 14, 2021.

(51) Int. Cl. A61B 8/08; A61F 2/10; G06T 7/00; G06T 7/60

(52) U.S. Cl. CPC A61B 8/5223; A61B 8/0858; A61F 2/105; G06T 7/0016; G06T 7/60; G06T 2207/10132; G06T 2207/30088; G06T 2207/30096

(57) ABSTRACT

A method is provided for treating a skin wound. In accordance with the method, an ultrasound image is obtained of a skin wound on a patient. The ultrasound image is processed to extract information that is correlated to a degree of wound healing. The degree of wound healing is assessed based at least in part on the extracted information. The skin wound is treated based at least in part based on the assessed degree of wound healing.

### CROSS REFERENCE TO RELATED APPLICATION

This application is a National Phase in the United States of PCT/US2022/012433, filed Jan. 14, 2022, which claims the benefit of U.S. Provisional Application No. 63/137,275, filed Jan. 14, 2021, the contents of both applications are incorporated herein by reference.

### GOVERNMENT FUNDING

This invention was made with government support under AG065776 awarded by the National Institutes of Health. The government has certain rights in the invention.

### BACKGROUND

Chronic wounds can be difficult to heal and are often accompanied by pain and discomfort. Multiple skin substitutes or Cellularized/Tissue based skin Products (CTPs) have been used to facilitate closure of complex wounds. Allografts from cadaveric sources have been a viable option in achieving such closure. However, early assessment of graft incorporation has been difficult clinically, often with delayed evidence of failure. Visual cues to assess graft integrity have been limited and remain largely superficial at the skin surface. Furthermore, currently used optical imaging techniques can only penetrate a few millimeters deep into tissue. Ultrasound (US) imaging offers a potential solution to address this limitation.

There are several wound treatment modalities. Standard wound care elements include serial debridement, compression therapy, judicious use of various wound dressings to control moisture balance, along with antimicrobial efforts to optimize the wound bed for successful healing. CTPs or bioengineered skin grafts made of synthetic and/or biological materials are often used as aids to promote wound closure and restore skin function. The allograft skin substitutes provide a scaffold to the open wound defect. This scaffold will then become colonized by host immune cells as the healing cascade progresses. Clinicians often refer to the process of graft integration with the recipient area as “taking”. Healthy host cells infiltrate the allograft matrix. These cells then differentiate into regenerated tissue ultimately integrating with surrounding healthy host tissue in a seamless fashion. Allograft take is typically dependent on the integrity of a healthy granular host wound base. A negative immune response, infection, excessive exudation, and compromised hemostasis can all endanger the survival of the allograft. Obstruction of adherence to granulating tissue and penetration of neo-capillaries can occur. Currently, clinicians rely on clinical experience and visual cues such as graft color, odor, texture, edema, drainage and necrosis to monitor the graft and the underlying wound health. However, visual inspection is limited to the skin surface whereas underlined edema and graft detachment can go unnoticed. Furthermore, studies have shown extensive heterogeneity in wound evaluations between different healthcare professionals. This underscores the necessity for a more objective wound assessment system that avoids this observational variation.

### SUMMARY

The use of ultrasound (US) to monitor wound healing and allograft integration has been evaluated to develop systems and techniques for improving monitoring, assessing and treating of wounds. A commercially available dual-mode (ultrasound and photoacoustic) scanner was employed which operates only in US mode. We compared the reported wound size from the clinic to the measured size using US in 45 patients. Two patients from this cohort received an allogenic skin graft and underwent multiple US scans over a 110-day period. All the data was processed by two independent analysts and one of them was blinded to the study. We measured change in US intensity and wound contraction as a function of time. The results show a strong correlation (R²=0.81, p<0.0001) between clinically and US measured wound sizes. Wound contraction greater than 91% was seen in both patients after skin grafting. An inverse relationship between wound size and US intensity (R²=0.77, p<0.0001) showed that the echogenicity of the wound bed increases as healthy cells infiltrate the allograft matrix regenerating and leading to healthy tissue and reepithelization. This work shows that US can be used to measure wound size and visualize tissue regeneration during the healing process.

In one particular aspect, a method is provided for treating a skin wound. In accordance with the method, an ultrasound image is obtained of a skin wound on a patient. The ultrasound image is processed to extract information that is correlated to a degree of wound healing. The degree of wound healing is assessed based at least in part on the extracted information. The skin wound is treated based at least in part based on the assessed degree of wound healing.

In accordance with another aspect, the extracted information is a measure of intensity of the ultrasound image.

In accordance with another aspect, the measure of intensity is inversely correlated with wound size.

In accordance with another aspect, the measure of intensity is a mean gray scale value of the ultrasound image.

In accordance with another aspect, treating the skin wound includes performing a skin graft.

In accordance with another aspect, the method further includes monitoring the treatment over time by obtaining and processing additional ultrasound images at subsequent times.

In accordance with another aspect, the method further includes determining that the skin wound is healing if the measure of intensity exacted from the additional ultrasound images increases over time.

In accordance with another aspect, the method further includes predicting wound deterioration due to tissue loss based on the monitoring

In accordance with another aspect, a method is provided for monitoring treatment of a skin wound. In accordance with the method, an ultrasound image is obtained of a skin wound on a patient. The ultrasound image is processed to extract information that is reflective of wound size. A degree of wound healing is assessed based at least in part on the extracted information. The skin wound is treated based at least in part based on the assessed degree of wound healing. The treatment is monitored over time by obtaining and processing additional ultrasound images at subsequent times.

In accordance with another aspect the extracted information that is reflective of wound size is a measure of intensity of the ultrasound image, the measure of intensity being inversely correlated with wound size.

In accordance with another aspect, the method further includes modifying the treatment of the skin wound or performing an additional treatment of the skin wound based on the monitoring.

This Summary is provided to introduce a selection of concepts in a simplified form. The concepts are further described in the Detailed Description section. Elements or steps other than those described in this Summary are possible, and no element or step is necessarily required. This Summary is not intended to identify key features or essential features of the claimed subject matter, nor is it intended for use as an aid in determining the scope of the claimed subject matter. The claimed subject matter is not limited to implementations that solve any or all disadvantages noted in any part of this disclosure.

### BRIEF DESCRIPTION OF THE DRAWINGS

FIG. 1A is a schematic representation of a typical ultrasound (US) scan over a wound site; FIG. 1B shows a typical B-mode US image showing coronal cross section of the lower limb; and FIG. 1C is a graph showing a comparison between reported wound width at the point-of-care and measured wound width using US.

FIGS. 2A-2C depict the wound progression and FIG. 2D shows the imaging and intervention/treatment timeline for patient A; FIGS. 2E-2G depicts the wound progression and FIG. 2H shows the imaging and intervention/treatment timeline for patient B.

FIGS. 3A-3B show a Bland-Altman analysis between measurements made by two independent analysts using the same set of images; FIGS. 3C-3F show the change in US intensity and wound area for patient A at sites i and ii; and FIGS. 3G-3L show the change in US intensity and wound size for patient B at sites i, ii and iii showing an 86.1%, 78.6% and 91.7% contraction respectively.

FIGS. 4A-4O show the differences between wound and healthy tissue over time.

FIGS. 5 and 5B are graphs showing the relationship between wound size and US intensity for patients A and B, respectively.

FIG. 6 shows a Bland-Altman analysis of reported wound width and (US) measured wound width in 45 patients.

FIGS. 7A-7P show the visual and US scan progression over two wounds and one healthy site.

FIGS. 8A-8Y show an ROI based analysis for Patient B of photographs and US images of sites i, ii, iii, and superior healthy tissue from Day 1 (FIGS. 8A-4E), Day 42 (FIGS. 8F-8J), Day 62 (FIGS. 8K-8O), Day 84 (FIGS. 8P-8T) and Day 106 (FIGS. 8U-8Y).

### DETAILED DESCRIPTION

### Introduction

Ultrasound (US) imaging is a unique imaging modality that addresses the aforementioned limitations of other imaging modalities. It is a quick, inexpensive, non-radiative, non-invasive, and point-of-care imaging modality that can look deep into soft tissue (up to 10 cm). (Ng and Swanevelder 2011, Szabo and Lewin 2013) The use of ultrasound as a therapeutic tool to aid debridement and promote healing has been widely reported but there are very few reports of US imaging being used to monitor wound healing. (Driver and Fabbi 2010, Ennis, et al. 2011, Kavros, et al. 2007) A recent small case series showed that US, doppler, and elastography could be used to determine wound morphology, biomechanics and proximity to other anatomical structures like bone and tendon. (Henshaw, et al. 2020) Another case study in a 46-year old female with a stage IV sacral pressure ulcer showed that multiple two-dimensional US images could be reconstructed for three-dimensional visualization of the ulcer. (Yabunaka, et al. 2015) Photoacoustic imaging and high-resolution harmonic US has also been used to stage and track healing of pressure ulcers and burn wounds in animal models. (Gnyawali, et al. 2015, Hariri, et al. 2019) Although these studies depict the advantages of US imaging like deeper imaging and elastography, a longitudinal human clinical study monitoring wound healing has not yet been reported. The ability to monitor a skin graft as it integrates would have high clinical significance and allow clinicians to make more informed therapeutic decisions. This work aims to evaluate the use of US to monitor wound healing and allograft integration.

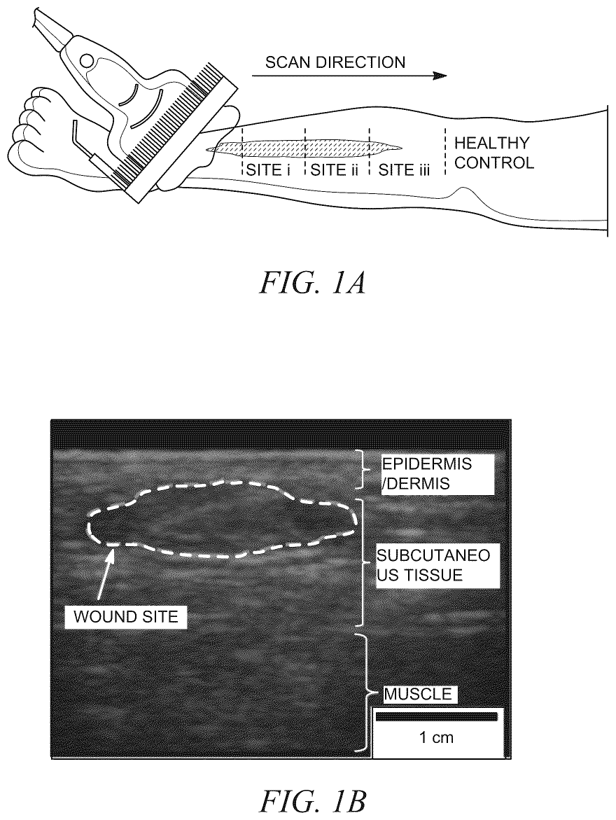

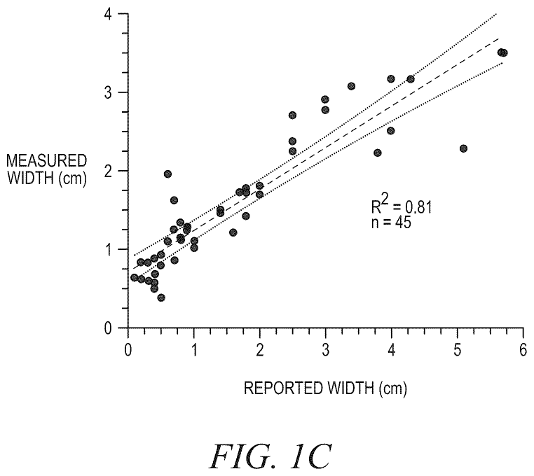

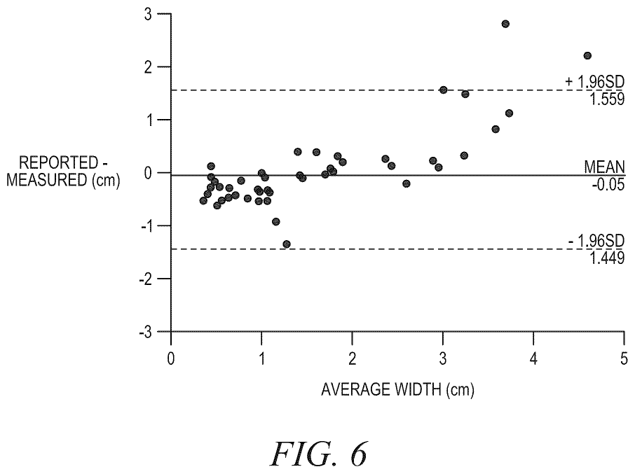

In this analysis 45 patients diagnosed with chronic foot wounds were scanned using ultrasound. Currently, wounds are sized by eye using a wound ruler as reference. Although more advanced sizing protocols using high-definition cameras using complex edge detection and neural networks have been reported, their limited penetration into tissue makes depth measurements difficult. Furthermore, wounds may prematurely contract at the surface level only ahead of a voluminous deeper cavity defect. This dimension is often neglected though it may further explain aberrant wound healing delays or cause of wound deterioration. The ability of US to non-invasively image under the skin surface make it an ideal imaging modality to solve such limitations. It will be demonstrated below (FIG. 1C) that US imaging can correctly size wounds of different sizes (R²=0.81). Statistically US measured wound size showed no clinically significant differences compared to the reported values from the clinic (FIG. 6). Using US, we could measure wound depth, but these are usually difficult to measure by eye and hence rarely reported.

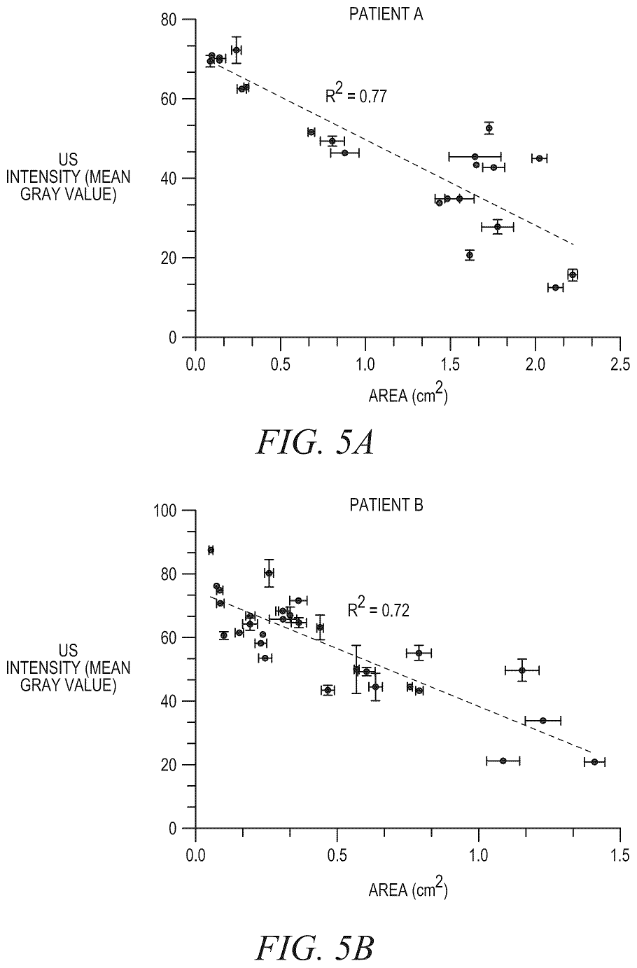

Furthermore, we show that US intensity is a marker of tissue regeneration (FIG. 5). The negative correlation between US intensity and wound area shows that as the skin graft takes, wound size reduces as healthy cells infiltrate the graft matrix. The infiltration results in tissue regeneration and an increase in US gray scale value.

One limitation is that wounds above 4 cm in width are consistently undersized. This is because the US transducer used in this study has 128-elements each measuring 3.5 mm long with a pitch of 0.3 mm, giving it a lateral field of view of ˜4 cm. Using a bigger transducer with more sensing elements or using a tomographic system can solve this issue.

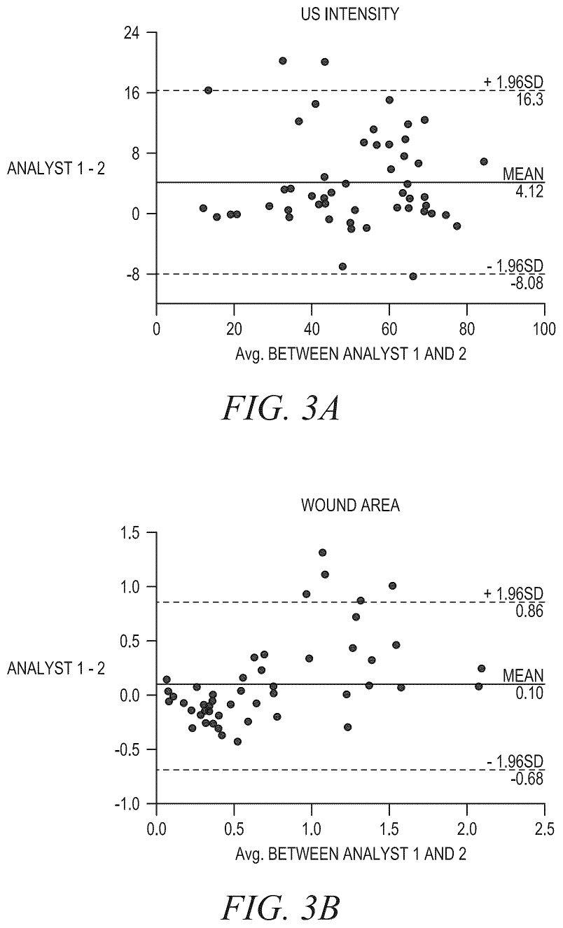

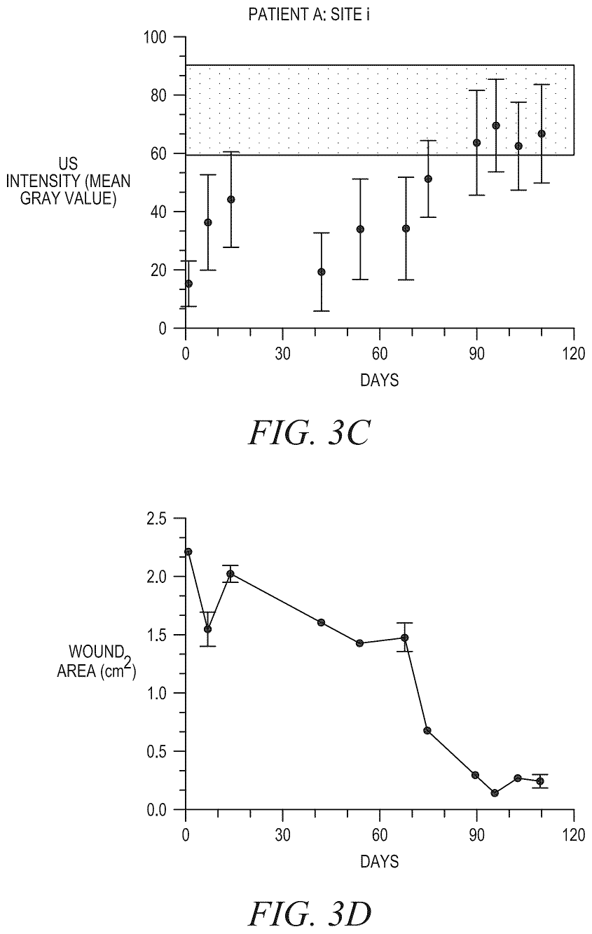

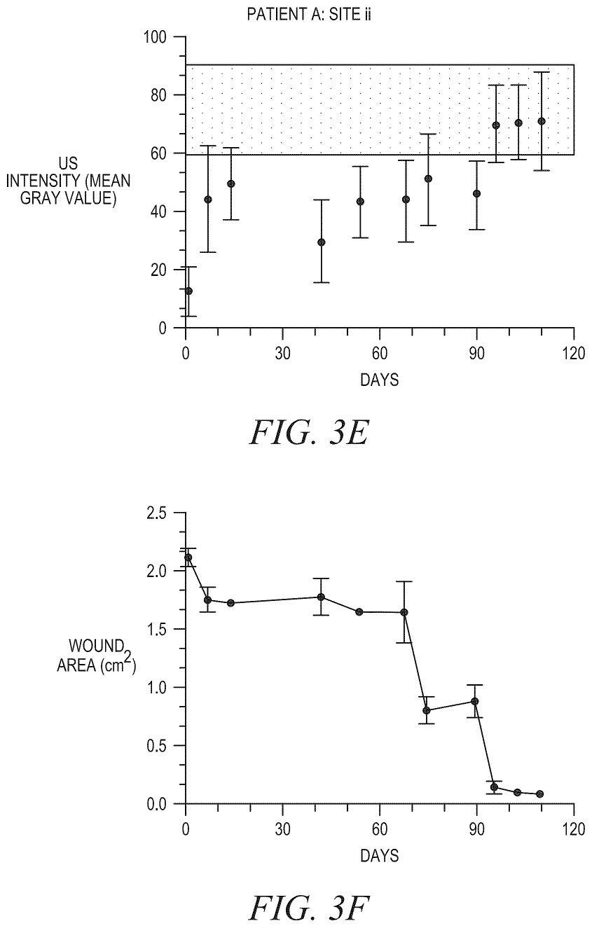

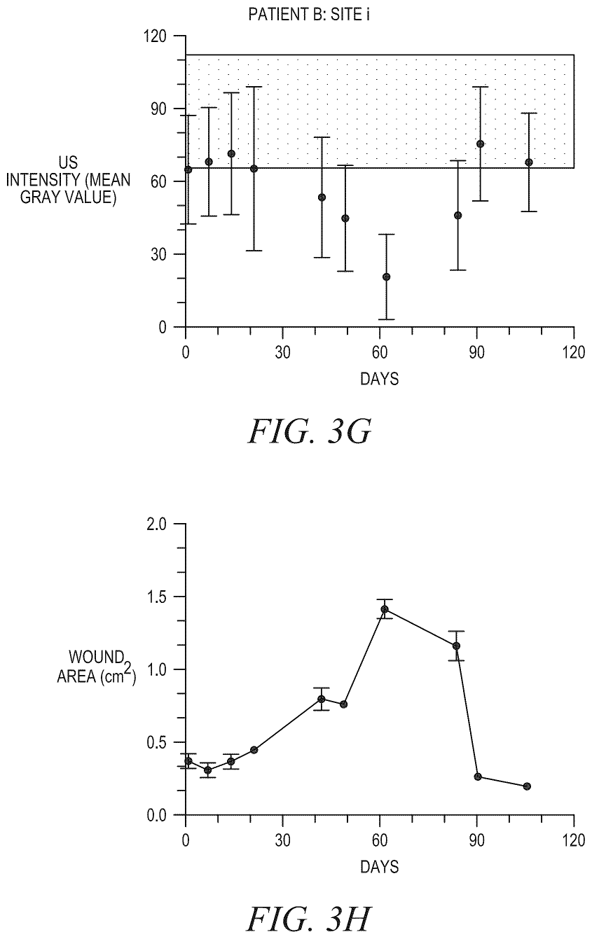

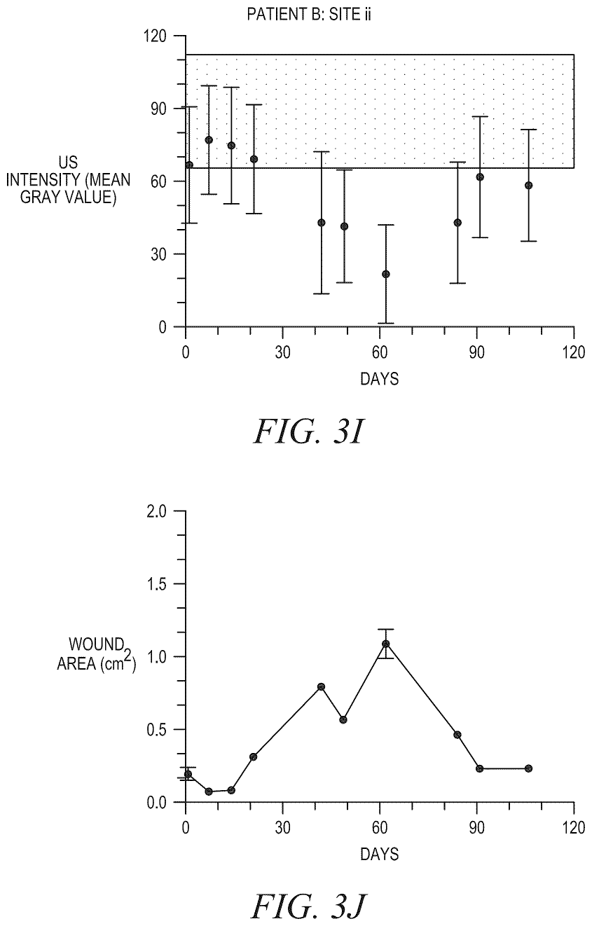

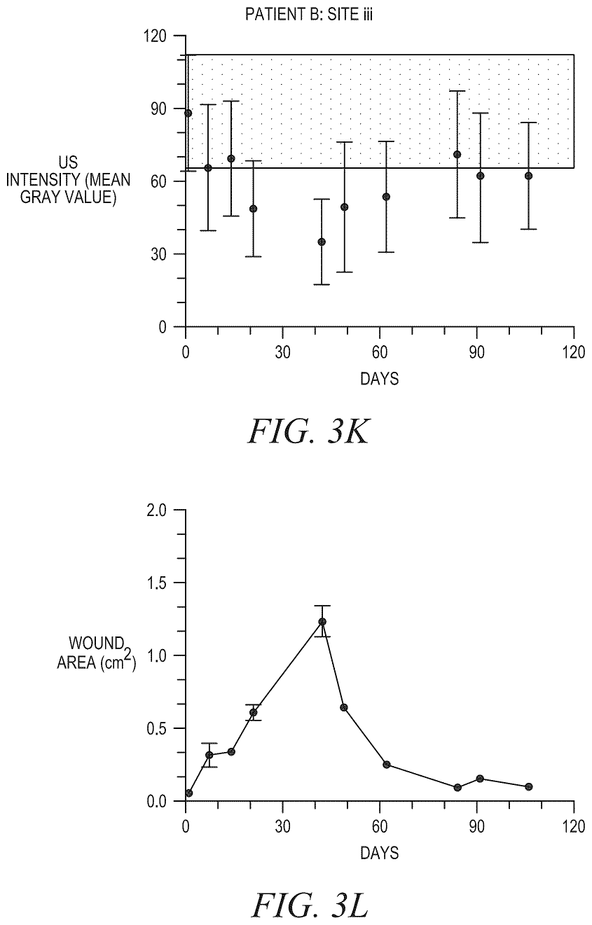

As further described below, we scanned 2 patients (referred as Patient A and B) who received allogenic skin grafts as part of normal wound treatment. Tissue loss or edema accumulation results in hypoechoic regions on US images. On the other hand, scar tissue and edema reduction through use of compression modalities results in hyperechoic regions.²⁰ Patients have unique soft tissue density and collagen arrangements giving them unique baseline acoustic properties. It is important to measure healthy tissue US intensity separately for each case to distinguish between diseased, healthy, and scar tissue. With skin grafts, the ideal outcome is to restore anatomical continuity along with functionality while minimizing scarring. US imaging is an effective tool to visualize tissue regeneration because US intensity increases as the wound heals. We showed the ability to define whether a wound was healed using the US intensity of the wound bed (FIG. 3). This in turn helps us monitor wound healing longitudinally. We defined a healthy tissue contrast window within one standard deviation of the dynamic baseline. US intensities falling under and over this window were considered as wound and scar tissue respectively.

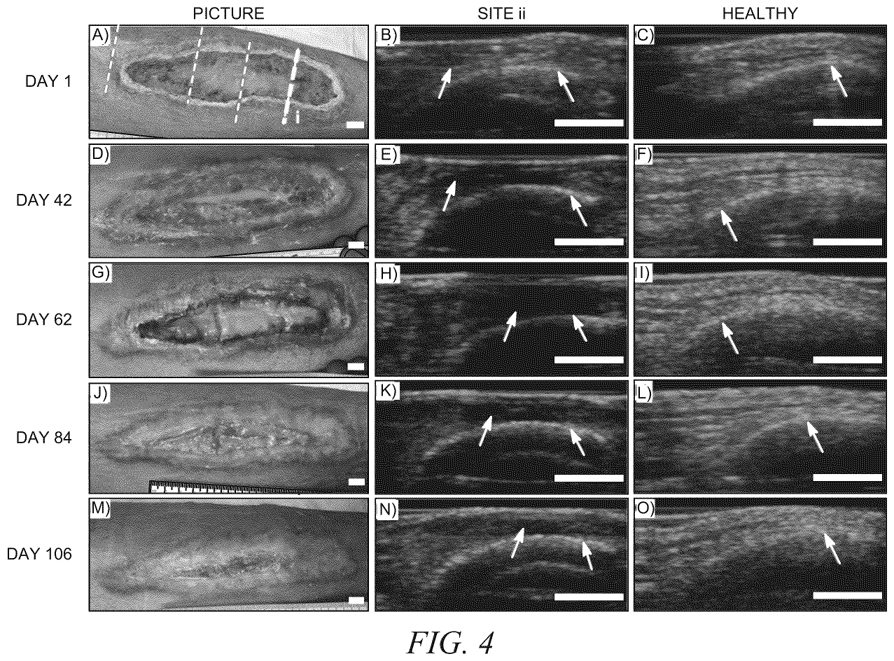

Sometimes wounds deteriorate secondary into a deeper unhealed voluminous defect. This defect often collects fluid and may evolve into a seroma or an abscess. Such a defect is particularly difficult to predict and visualize with current methods at the bedside. Therefore, visual clinical cues such as erythema, swelling, fluctuance and/or presence of pain can be used to predict tissue health under the skin. We show that US imaging is especially effective in providing insight into deeper wound elements by predicting tissue loss as early as Day 1 (FIG. 4).

Also demonstrated below is the ability to monitor wound health after skin grafting. Once skin grafted, the wound surface is completely obscured from the clinician's view, and the top layer of then graft often dies due to lack of blood flow. It can take weeks for doctors to diagnose a non-taking skin graft whereas we show that we can visualize tissue regeneration longitudinally. This is visible in Patient B who was grafted on day 49, followed by a period of tissue loss after skin grafting and then a rapid healing. It is key to note that different areas of the wound responded differently to skin grafting. Site iii responded before sites I and ii (FIGS. 3H, 3J and 3L).

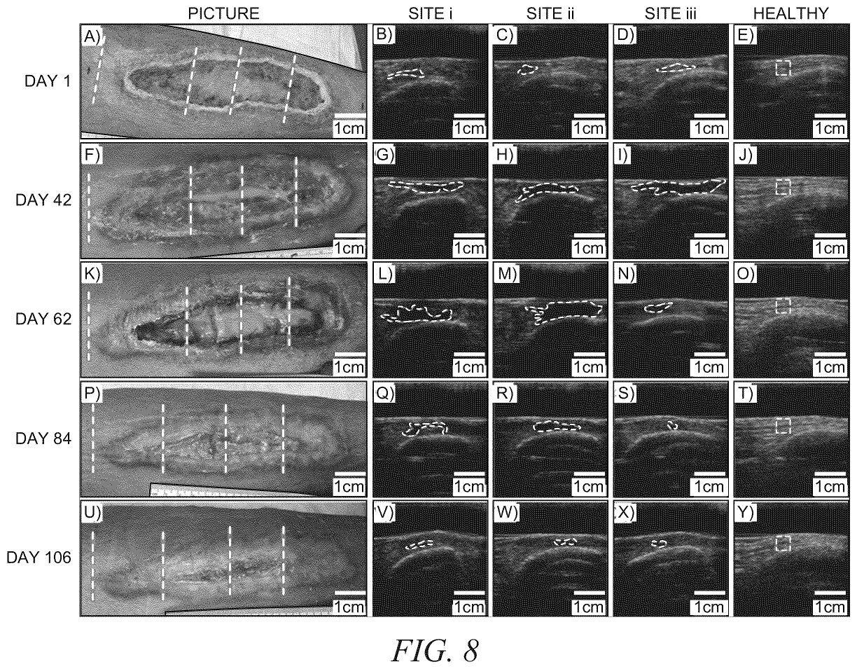

The ability of US to predict tissue loss is seen in patient B (FIG. 4) where site ii shows signs of tissue loss approximately 0.5 cm under the wound surface on day 1 (FIGS. 4B and 8). This loss in tissue is obscured form the clinicians as it starts from the inside-out. The evidence of tissue loss initiates the need for early and advanced therapy, in this case skin grafting on Day 49. The hypoechoic regions between the tibia and the US probe cover shown below in FIGS. 4E and 4H are indicative of further tissue loss which left the tibia exposed by day 42. The increase in wound size and loss of tissue was also corroborated by image analysis (FIGS. 3G-3L) where US intensity reduces, and wound area increases over the first 62 days. After grafting the wound heals rapidly that is monitored as well.

The lack of healing in Patient A initiated the use of a second allogenic skin graft. This lack of healing is usually diagnosed by years of experience, but longitudinal US imaging can quantify it objectively (FIGS. 3C-3F).

Additional details and results concerning this study will be presented below.

### Materials and Methods

### Patients

Patient inclusion criteria were as follows: (i) patients with ulcers not more than 15 cm² in area. ii) patients 18 years or older and be able to provide consent. Exclusion criteria included i) Patients with a blood-borne pathogen ii) patients with other lesions (e.g., Melanomas) at the wound site. iii) patients with orthopedic implants patients presenting with wounds between digits or in the pubic region; 63 patients (65 wounds) were recruited for this study. Table 1 describes the patient demographic distribution. All patients were imaged.

TABLE 1
Patient demographic distribution.

| Category | Distribution |
|---|---|
| Age (mean ± standard deviation) | 66.9 ± 14.6 |
| Male/Female | 31/32 |
| Diabetic/Non-diabetic | 19/44 |
| Chronic venous hypertension (Yes/No) | 37/26 |

Two patients (Patients A and B) who received allogenic skin grafts underwent multiple follow-up scans over a period of 110 days. Patient A was a 77-year-old non-diabetic female with a history of hypothyroidism. Patient A presented with an open, chronic, post-operative left knee wound with tendon involvement and chronic venous hypertension on both sides. Patient A was first scanned 103 days after presentation followed by 13 follow-up scans over a 110-day period. Patient B was a 33-year-old non-diabetic male with a history of renal transplant. Patient B presented with a left, anterior, lower limb, posttraumatic ulcer with chronic venous hypertension on the left side. Patient B was first scanned 33-days after presentation followed by 10 follow-up scans over a 106-day period.

This study did not involve any additional visits and was performed during a regularly scheduled wound care visit. The frequency of visit was independently decided by the wound specialist (CA) depending on the patient's needs. The wound site was prepared by removing any dressings and cleaning with sterile saline prior to scanning. Neighboring tissue was further cleaned using alcohol swabs to prevent infection. A photograph with a wound ruler was also taken to correctly size the wound surface. To prevent cross contamination and infections, we used a new sterile CIV-Flex™ transducer cover for every scan (#921191 from AliMed Inc., Dedham, MA, USA).

### Ultrasound Imaging

We used a commercially available LED-based photoacoustic/ultrasound imaging system (AcousticX from CYBERDYNE Inc. (Tsukuba, Japan)). (Hariri, et al. 2018) In this study, we only used the ultrasound mode. The ultrasound transducer has 128 linearly arranged elements operating with a central frequency of 7 MHz, bandwidth of 80.9% and field of view of 4 cm. Sterile US gel (Aquasonic 100, Parker Laboratories Inc., Fairfield, NJ, USA) and a custom hydrophobic gel pad from CYBERDYNE INC. (Tsukuba, Japan) was used for US coupling between the transducer and skin surface. All US images were acquired at 30 frames per second.

Wounds smaller than 5 cm in length (Patient A) were scanned in a single sweep from inferior healthy tissue over the wound to superior healthy tissue. From these scans, we selected representative frames for inferior healthy tissue, two wound sites (site I and ii), and superior healthy tissue. For large wounds (Patient B), we selected three wounded sites at discrete distances from the inferior wound edge and tracked wound progression over time. FIG. 1A is a schematic representation of a typical ultrasound scan over a wound site. Small wounds (<5 cm in length) were scanned in one sweep from inferior to superior healthy tissue. Large wounds were imaged at discrete distances from the inferior wound edge. Superior and inferior tissue from the wound edge were considered as healthy controls. To monitor tissue regeneration, we measured changes in US intensity (e.g., mean gray value) and change in wound size as a function of time. All the images were acquired by YM only. It is important to note that all US scans were performed by hand. Thus, it is impossible to perfectly match US frames from the same site over multiple imaging sessions. To reduce the effects of this limitation, we matched the underlying bone morphology to compare similar spots over time. Patients with skin grafts went through the same imaging protocol with no added steps.

### Image Processing

All US frames were reconstructed and visualized using the AcousticX photoacoustic imaging system developed by CYBERDYNE Inc. (Tsukuba, Japan) version 2.00.10. B-mode, coronal cross-section images were exported as 8-bit gray scale images. The images were further processed to measure wound width, area, and US intensity using Fiji an ImageJ extension version 2.1.0/1.53c. (Schindelin, et al. 2012) Data was plotted using Prism9 version 9.0.0. All measurements were made using custom region of interest (ROI) analysis for individual frames. Drawing ROIs by hand can be very subjective. Hence, all image quantification was independently confirmed by two analysts (YM and JT). JT was blinded to the study and only received B-mode US images to analyze the data.

To corroborate our method with the current gold standard eye measurements we compared reported wound size with size measured with US imaging. The reported wound size was provided by CA and his nursing team and was recorded as part of standard care. The wound width under US was measured using custom ROI analysis to outline the wound on a single frame. Wound width was defined as the widest region within the wound ROI. FIG. 1B shows a typical B-mode US image showing coronal cross section of the lower limb. Wound width was measured at its widest point within the wound site (blue dotted line).

We quantified changes in US intensity over time in a constant ROI. A custom ROI (outline of wound site) was drawn for Patient A and B on day 1 and the US intensity within that ROI was tracked over time. Maintaining a constant ROI allows us to quantify tissue regeneration as the graft integrates into the wound bed. To further compare US intensity between wounded and healthy tissue, a dynamic baseline US intensity for healthy tissue was measured. We measured the mean gray value and its standard deviation (0.5 cm×0.5 cm ROI) in two frames representing inferior and superior healthy tissue, for each time point. The healthy window was defined within one standard deviation of the dynamic baseline. US intensities falling under and over this window were considered as wound and scar tissue respectively.

Finally, we monitored change in wound area under the skin graft. Custom ROIs were drawn for individual sites at each imaging time point. An area was considered to be a wound if its US intensity was significantly below the healthy window defined above.

### Statistical Testing.

A Bland-Altman analysis was used to look for bias between clinically reported wound (gold standard) and US measured wound width. A mean bias more that 0.06 cm would be considered clinically significant as the lateral resolution of the US transducer ranges from 0.05-0.06 cm.

To reduce the subjective nature of ROI-based analysis two independent analysts, YM and JT processed all the images. JT was blinded to the study. A Bland-Altman analysis was used to look for systemic bias between two independent analysis of the same images. For US intensity measurements a mean bias above 17 gray scale values (standard deviation in healthy tissue) would be considered clinically significant. For wound area measurements, a mean bias greater than 5% of the largest wound size would be considered clinically significant.

### Results

FIG. 1A shows a typical US scan over a wound site. We show that US imaging can be used to reliably size wounds only limited by the width of the transducer. Skin graft integration, tissue regeneration, and changes in wound size can be easily visualized and quantified using US, which is not possible by eye. We also show that there is a significant negative correlation (R²=0.77, p<0.0001) between wound size and US intensity.

Of the cohort of 63 patients with 65 wound sites, 20 wounds could not be sized with US: 15 wounds did not have a reported wound size from the clinic and were excluded; five wounds were located in regions with high curvature such as the lateral side of the toe or ankle, which makes US coupling difficult in these regions with a linear array transducer. Hence, 45 wounds were used to compare reported wound size with the US measured size. FIG. 1c is a graph showing a comparison between reported wound width at the point-of-care and measured wound width using US (n=45). The dotted lines represent 95% confidence intervals. The graph shows that there is a strong correlation (R 2=0.81, p<0.0001) between reported and measured wound widths. A Bland-Altman analysis shown in FIG. 6 was used to check for any bias between the gold standard eye measurements and our US imaging technique. A calculated mean bias of −0.05 cm rejected our null hypothesis of clinical significance above 0.06 cm.

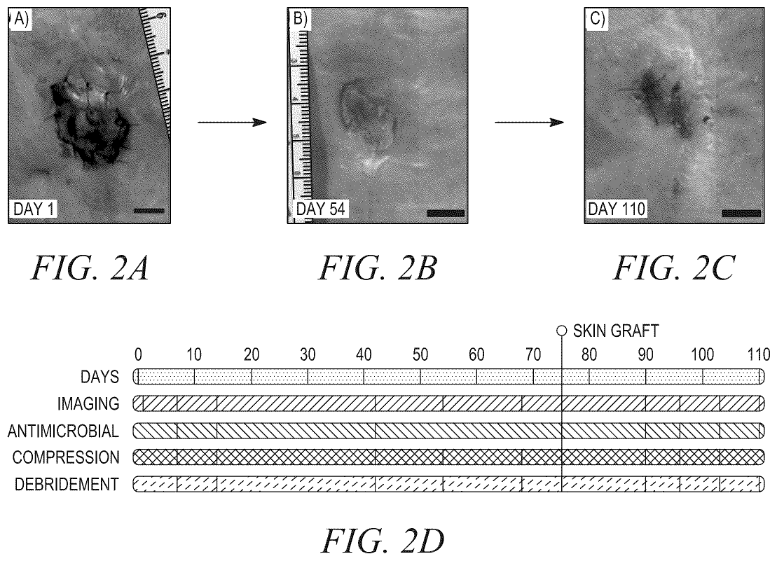

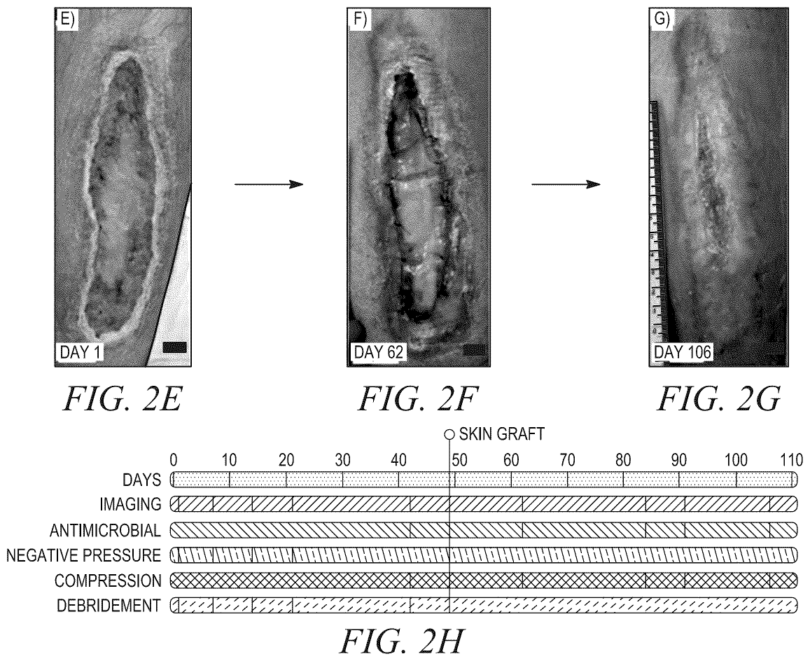

FIGS. 2A-2C depict the wound progression and FIG. 2D shows the imaging and intervention/treatment timeline for patient A. FIGS. 2E-2G depicts the wound progression and FIG. 2H shows the imaging and intervention/treatment timeline for patient B. All scale bars are 2 cm.

Patient A

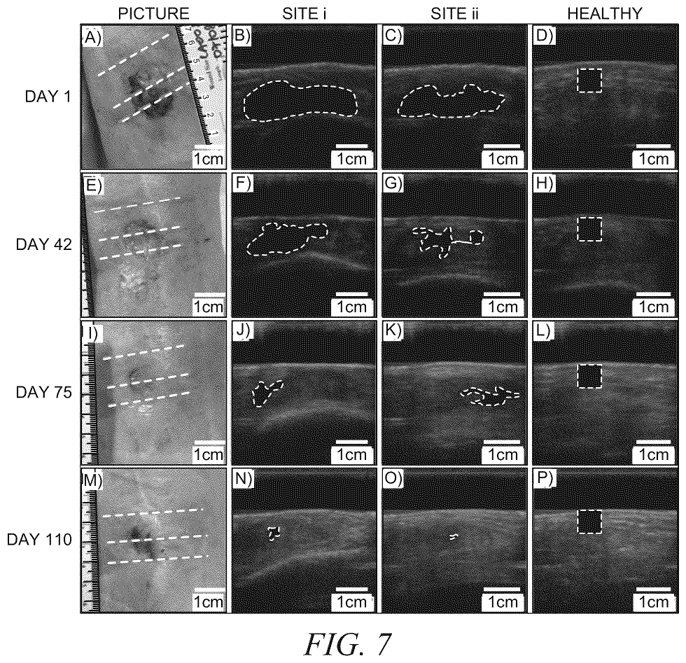

Patient A had a left knee ulceration who underwent 13 US scans over 110-day-scan. Patient A received a human cadaver-derived, acellular dermal matrix (AlloDerm, Lifecell, Branchburg, NJ) skin graft 28 days prior to the first scan. A second tissue matrix allograft composed of dehydrated human amnion/chorion membrane (EpiFix, MiMedx, Marietta GA) was used 75-days after the first scan. FIGS. 7A-7P show the visual and US scan progression over two wounds and one healthy site. Imaging sites are marked i, ii, and healthy from inferior-superior regions. Wound pictures (FIGS. 2A-2C) show wound contraction and skin graft integration at the surface over time. An increase in tissue echogenicity is seen at both sites i and ii indicative of skin graft integration (FIGS. 7A-7P). The superior healthy control tissue remains unchanged over the study period.

FIGS. 3A-3B show a Bland-Altman analysis between measurements made by two independent analysts using the same set of images. Statistical analysis of both US intensity (FIG. A) and wound area (FIG. 3B) showed no significant bias between the two analysts. FIGS. 3C-3F show the change in US intensity and wound area for patient A at sites i and ii. Site i and ii showed an 89.2% and 96.0% reduction in size respectively. FIGS. 3G-3L show the change in US intensity and wound size for patient B at sites i, ii and iii showing an 86.1%, 78.6% and 91.7% contraction respectively. Error bars in panels C, E, G, I, and K, represent standard deviation of mean gray value in a single ROI. Error bars in panels D, F, H, J, and L represent standard deviation of area measured using 3 US frames.

For Patient A, the dynamic baseline for healthy tissue was measured over 13 independent scans. (73.5±12.9 (unique for patient A)). Changes in US intensity over time for site i and ii showed that it took 90 days for site i and 96 days for site ii to achieve gray scale values similar to healthy tissue (FIGS. 3C and 3E). Wound size under the skin graft showed varied rates of contraction for both sites. Site i showed an 89.2% and site ii showed a 96.0% reduction in size over 110 days (FIGS. 3D and 3F). Wound contraction was slowest between days 14-68, which is consistent with the physician notes reporting slow improvements and signs of edema during the same period. Interestingly, wound contraction was fastest between days 68-90 which was also noted by the attending doctor.

Patient B

Patient B was a 33-year-old male with an anterior, lower left limb ulceration who underwent 10 US scans over a 106-day period. Patient B received a human cadaver-derived, acellular dermal matrix (AlloDerm, Lifecell, Branchburg, NJ) skin graft 49 days after the first scan. We monitored wound progression at three wound sites i, ii, and iii (inferior, medial, and superior, respectively). FIG. 4 shows the differences between wound and healthy tissue while focusing on site ii and superior healthy control only. FIG. 8 shows Annotated images on sites i and iii.

In particular, FIGS. 4A-4O show photographs and US images from Site ii and superior healthy tissue. FIGS. 4A-4C are from Day 1, FIGS. 4D-4F are from Day 42, FIGS. 4G-4I are from Day 62, FIGS. 4J-4L are from Day 84, and FIGS. 4M-4O) are from Day 106. Signs of tissue loss (FIG. 4B) can be seen using US while not visible to the eye (FIG. 4A). An increase in wound size, and hypoechoic regions were observed (FIGS. 4B, 4E, and 4H) over the first 62 days indicating tissue loss even though skin grafting was done on day 49. An increase in echogenicity indicating tissue regeneration and wound contraction can be seen between days 62-106 (FIGS. 4K and 4N). The ability to monitor tissue loss and regeneration under a skin graft between days 49-62 and 62-106 respectively, shows the main power of imaging over current methods. Superior healthy control tissue remains fairly unremarkable and unchanged over the same time period. All scale bars in FIG. 4 are 1 cm.

FIG. 8 shows an ROI based analysis for Patient B. The photographs and US images of sites i, ii, iii, and superior healthy tissue shown in FIGS. 8A-8E are from Day 1, in FIGS. 8F-4J from Day 42, in FIGS. 8K-8O from Day 62, in FIGS. 8P-8T from Day 84, and in FIGS. 8U-8Y from Day 106. A consistent increase in wound size, and loss of tissue is seen in all three wound sites, whereas superior healthy tissue remains the same. After skin grafting, site iii starts healing first (FIG. 8N) whereas site i and ii still show further tissue loss (FIGS. 8L-8M). This is particularly difficult to see by eye. By Day 84 and 106, an increase in US signal within the wound site shows that the skin graft has taken and wound size under the graft has reduced considerably for sites i, ii and iii. Superior healthy tissue (control) remains unchanged over the entire study period.

The average healthy gray value measured over 10 separate scans for Patient B was 90.4±21.5 (unique for patient B). All wound sites showed an increase in wound size and loss of US signal until skin grafting followed by a tissue regeneration and wound contraction period. Healthy control tissue remained unchanged over the same time period. Sites i and ii responded differently compared to site iii (FIGS. 3G-3L). Between days 1-62, the wound area at site i increased from 0.37 cm² to 1.42 cm² while Site ii increased from 0.20 cm² to 1.10 cm². Both sites i and ii showed an increase in wound size and tissue loss up to 62 days into the study even though patient B was skin grafted on day 49 (FIGS. 3 H and J). Site iii responded to treatment at a different rate. Wound area at site iii increased from 0.05 cm² to 1.23 cm² within the first 42 days followed by a healing period. By the end of the study, sites i, ii, and iii showed an 86.1%, 78.6%, and 91.7% contraction respectively. Photographic pictures clearly show that Patient B's wound had not completely healed by the end of the study (FIG. 4M). Incomplete healing can be seen when US intensity was monitored over time (FIGS. 3G-3K). The measured US intensities approach the healthy window but do not show complete regeneration after 110 days of treatment.

A plot of wound area vs. US intensity, shown in FIG. 5A for patient A and FIG. 5B for patient B, revealed an inverse relationship between the two variables with R²=0.77 and 0.72 for patients A and B, respectively. A large wound with less tissue showed a low US signal while a small wound with regenerated tissue showed a high US signal.

All data quantification was done by two independent analysts (YM and JT). JT was blinded to the study while YM acquired and analyzed all the data. Bland-Altman analysis revealed no significant bias between the two analysts. For US intensity measurements, a mean bias of 4.12 gray values between the two analysts, rejected our null hypothesis of clinical significance above 17 gray values (standard deviation in healthy tissue). For wound area measurements, a mean bias of 0.10 cm² (4.4% of largest measured wound size) rejected our null hypothesis of clinical significance above 5% of the largest measured area.

### Discussion

The fast, inexpensive, reliable, and pain free characteristics of point-of-care US imaging make it an ideal modality to monitor wound progression. Currently, wounds are sized by eye using a wound ruler as reference. Although more advanced sizing protocols using high definition cameras using complex edge detection and neural networks have been reported, their limited penetration into tissue makes depth measurements difficult. (Chino, et al. 2020, Malone, et al. 2020, Toygar, et al. 2020) Furthermore wounds may prematurely contract at the surface level only ahead of a voluminous deeper cavity defect. (Medina, et al. 2005) This dimension is often neglected though it may further explain aberrant wound healing delays or cause of wound deterioration. The ability of US to non-invasively image under the skin surface make it an ideal imaging modality to solve such limitations. FIG. 1C shows that wound width measured using US imaging has strong correlation (R²=0.81) with the reported eye measured values. The Bland-Altman analysis also showed no clinically significant difference between reported and measured values (FIG. 6). One limitation is wounds above 4 cm in width are consistently undersized. This is because the US transducer used in this study has 128-elements each measuring 3.5 mm long with a pitch of 0.3 mm, giving it a lateral field of view of about 4 cm. (Hariri, et al. 2018) US imaging has value at estimating wound depth, but these are usually difficult to measure by eye and hence are rarely reported.

Tissue loss or edema accumulation results in hypoechoic regions on US images. (Ripollés, et al. 2013, Terslev, et al. 2003) On the other hand, scar tissue and edema reduction through use of compression modalities results in hyperechoic regions. (Ackerman, et al. 2019) Patients have unique soft tissue density and collagen arrangements giving them unique baseline acoustic properties. It is important to measure healthy tissue US intensity separately for each case to distinguish between diseased, healthy, and scar tissue. With skin grafts, the ideal outcome is to restore anatomical continuity along with functionality while minimizing scarring. US imaging is an effective tool to visualize tissue regeneration because US intensity increases as the wound heals. This phenomenon is clearly visible with both patients A and B. FIG. 3 shows the change in US intensity and wound size over time. It is interesting to note an inverse relationship between wound size and US intensity for both patients (FIGS. 5A and 5B). In both patients, the corresponding US intensity increases whenever wound size reduces. This is because healthy cells infiltrate into the graft matrix regenerating healthy tissue as the skin graft takes; this in turn increases US signal. Skin grafting did not lead to scar formation because the US intensity of regenerated tissue stayed within the defined healthy tissue window (FIGS. 3C, 3E, 3G, 3I, and 3K). A high collagen orientation index in scar and keloid tissue leads to hyper-echogenicity under US imaging. (Timar-Banu, et al. 2001, Verhaegen, et al. 2009)

Sometimes wounds deteriorate secondary to a deeper unhealed voluminous defect. This defect often collects fluid and may evolve into a seroma or an abscess. (Landis 2008) Such a defect is particularly difficult to predict and visualize with current methods at the bedside. Therefore, visual clinical cues such as erythema, swelling, fluctuance and/or presence of pain can be used to predict tissue health under the skin. (Ren, et al. 2020) Ultrasound imaging is especially effective in providing insight into deeper wound elements. The real time, high-resolution, high-depth penetration nature of ultrasound allows it to detect signs of edema and tissue loss before it is visible on the skin surface. The ability of US to predict tissue loss is seen in patient B (FIG. 4) where site ii shows signs of tissue loss approximately 0.5 cm under the wound surface on day 1 (FIGS. 4B and 8). The hypoechoic regions between the tibia and the US probe cover in FIGS. 4E and 4H are indicative of further tissue loss which left the tibia exposed by day 42. The increase in wound size and loss of tissue was also corroborated by image analysis (FIGS. 3G-4L) where US intensity reduces, and wound area increases over the first 62 days.

Wound healing is an extremely complicated process that is affected by many variables such as age, local perfusion, moisture and patient compliance. (Guo and DiPietro 2010, Valencia, et al. 2001) Younger patients are more likely to heal faster than older patients. (Gould, et al. 2020) Comparing the healing rates between patient A (77-year old) and patient B (33-year-old) in FIG. 3 shows that patient B exhibited faster wound contraction after skin grating compared to patient A. Patient A showed a 62% contraction within 103 days of the first skin graft placement. Patient B had a larger wound but 65% contraction within 42 days of skin grafting. Furthermore, patient A required a secondary skin graft (EpiFix, MiMedx, Marietta GA) 54 days into the study after which the wound contraction rate increased at both sites (FIG. 3 C-F). These results suggest that US imaging could be used to evaluate wound healing rate; however, a more comprehensive and well powered study is needed to draw conclusions.

In summary, ultrasound imaging is a valuable tool to study skin graft integration used for chronic wound treatment. We first showed that wound width measurements made by US imaging are strongly correlated to measurements made visually. Second, a thorough investigation into two patients (5 wound sites), both receiving allogenic skin grafts showed that wound contraction, healing, and skin graft integration could be monitored using wound area and US intensity. The loss of tissue and inflammation resulted in hypoechoic regions on the ultrasound which healed after skin graft placement and integration in both patients. Over 110 days, patient A showed up to 96% wound contraction whereas patient B showed up to 91.7% contraction over a similar period. During the healing process, US intensities recovered into the healthy tissue window showing soft tissue regeneration under the skin graft.

What is claimed is:

1. A method for treating a skin wound, comprising:
obtaining an ultrasound image of a skin wound on a patient;
processing the ultrasound image to extract information that is correlated to a degree of wound healing;
assessing the degree of wound healing based at least in part on the extracted information; and
treating the skin wound based at least in part based on the assessed degree of wound healing.

2. The method of claim 1 wherein the extracted information is a measure of intensity of the ultrasound image.

3. The method of claim 2 wherein the measure of intensity is inversely correlated with wound size.

4. The method of claim 2 wherein the measure of intensity is a mean gray scale value of the ultrasound image.

5. The method of claim 1 wherein treating the skin wound includes performing a skin graft.

6. The method of claim 1 further comprising monitoring the treatment over time by obtaining and processing additional ultrasound images at subsequent times.

7. The method of claim 6 further comprising determining that the skin wound is healing if the measure of intensity exacted from the additional ultrasound images increases over time.

8. The method of claim 6 further comprising predicting wound deterioration due to tissue loss based on the monitoring.

9. The method of claim 1 wherein obtaining an ultrasound image of a skin wound on a patient including performing an ultrasound scan of the skin wound.

10. A method of monitoring treatment of a skin wound, comprising:
obtaining an ultrasound image of a skin wound on a patient;
processing the ultrasound image to extract information that is reflective of wound size;
assessing a degree of wound healing based at least in part on the extracted information;
treating the skin wound based at least in part based on the assessed degree of wound healing; and
monitoring the treatment over time by obtaining and processing additional ultrasound images at subsequent times.

11. The method of claim 10 wherein the extracted information that is reflective of wound size is a measure of intensity of the ultrasound image, the measure of intensity being inversely correlated with wound size.

12. The method of claim 10 further comprising modifying the treatment of the skin wound or performing an additional treatment of the skin wound based on the monitoring.

13. The method of claim 1 wherein obtaining an ultrasound image of a skin wound on a patient including performing an ultrasound scan of the skin wound.
